(19) United States
(12) Patent Application Publication
(10) Pub. No.: US 2021/0290223 A1
(43) Pub. Date: Sep. 23, 2021

(54) DEVICE AND METHODS FOR USE IN ROBOTIC ASSISTED SURGERY FOR TREATMENT OF OBSTRUCTIVE SLEEP APNEA

(71) Applicant: Zelegent, Inc., La Jolla, CA (US)

(72) Inventors: Yosef Krespi, New York, NY (US); Ron Hadani, Herzeliya (IL); Alexander Arrow, Lakeside, CA (US); Richard Lilly, San Jose, CA (US)

(21) Appl. No.: 17/153,873

(22) Filed: Jan. 20, 2021

Related U.S. Application Data
(60) Provisional application No. 62/964,093, filed on Jan. 21, 2020.

(51) Int. Cl. A61B 17/04; A61B 17/06; A61B 34/30

(52) U.S. Cl. CPC A61B 17/0482; A61B 17/06166; A61B 2017/06176; A61B 34/30; A61B 2017/06171; A61B 17/06066

(57) ABSTRACT

There is provided a suture passer device used in procedures of robotic assisted minimally invasive suspension of the hyoid and tongue base in treatment of sleep apnea. The suture passer device is a dual use suture passer, having both suture insertor and extractor functionality on a single unitary instrument section of the device by means of insertion and extraction hooks positioned on an elongated needlelike body which is connected to a handle grip. The insertion and extraction hooks receive a suture via an opening in the body of the instrument, with the suture capable of removal and fastening to the extraction hook by robotic forceps. Procedures for using the device are disclosed which include a single point of entry on a patient and the device moved to first and second positions above and below the hyoid bone during robotic assisted surgery.

### CROSS REFERENCE TO RELATED APPLICATIONS

This application claims the benefit of and priority to U.S. Provisional Application No. 62/964,093, filed on Jan. 21, 2020 and incorporated by reference herein in its entirety.

### FIELD OF THE INVENTION

The present invention relates to a suture passer device for use in robotic assisted surgery in the treatment of obstructive sleep apnea and methods of using the device.

### BACKGROUND

Obstructive sleep apnea (OSA) is a well-known problem. Sleep disorders cost both people and corporations financially in lost productivity and apnea has similar risk factors for heart disease as other known problems such as smoking, high cholesterol and alcohol. OSA is a disruption of more than 10 seconds between breaths during sleep with more than 10 episodes per hour. With hypertension patients, 5 episodes per hour may be defined as OSA.

OSA is a large and growing problem worldwide and is an increasing problem with correlations to overweight and obesity as well as increasing age. It is also associated with expensive medical care, including post-surgical and post-procedural complications and longer hospital stays.

When OSA is diagnosed, treatment is often prescribed on four levels of ascending complexity: a.) behavioral changes, weight loss or modified drinking, smoking or sleeping habits; b.) CPAP pump and mask to assist in sleep breathing; c.) oral appliances if CPAP is not tolerated by the patient; and d.) surgical procedures if the above are not successful.

The common surgical procedures for OSA include nasal surgery such as septum, turbinate reduction, swell bodies; oro-pharyngeal; and tongue base which includes tongue reduction, hypoglossal nerve stimulation, and hyoid suspension. Prior tongue and hyoid suspension and expansion surgical techniques have been invasive surgeries. The procedures typically require more than one surgical entry point on a patient and provide little maneuverability and visibility for the surgeon during the process for handling the sutures needed to suspend the tongue and hyoid.

### SUMMARY

The present invention is a suture passer device used in procedures of robotic assisted minimally invasive suspension of the hyoid. The suture passer device of the present invention is a dual use suture passer, having both suture insertor and extractor functionality on a single unitary device. That is, insertion and extraction are all performed with one instrument on the device of the present invention. The suture passer device of the present invention is a static needle with two directional hooks to achieve insertion and extraction under robotic visualization. This is accomplished without any spring mechanism to catch the suture and also without changing the diameter of the instrument (ie. an alligator tip).

The suture passer device of the present invention includes an elongated needle like instrument having a body with an external surface, a first end received by a handle section and a second end with a needle tip. The instrument has an insertion hook for securing and delivering a suture during insertion and also an extraction hook for receiving and securing a suture during extraction. The insertion hook and the extraction hook are defined by an open mouth area located between the insertion hook and the extraction hook, and an internal surface extending from the external surface of the body adjacent to the insertion hook to the external surface of the body adjacent to the extraction hook, thereby forming an opening in the body of the suture passer device which receives the suture when the suture is placed into the mouth area on the instrument. The insertion hook receives and secures the suture for delivery of an insertion of the suture by the instrument and the extraction hook receives and secures the suture for extraction of the suture during extraction by the instrument. The suture passer device includes a head section angled away from a central axis of the instrument body of the device. The body of the instrument has a cylindrical or oval shape, but other shapes are within the scope of the present invention.

With the suture passer device of the present invention the insertion hook is located on the instrument body at a position proximal to the handle section relative to the extraction hook.

The suture passer device of the present invention allows for an alternative to tongue based surgery. The procedure with the device of the present invention provides for robotic guided surgery with improved accuracy, reach and visualization in the surgical area.

The procedure is facilitated by the dual use suture passer (insertor-extractor) device of the present invention and includes use of barbed sutures. There is also a special design tongue blade for exposure and safety. The suspension of tissue is powered by special barbed sutures, composed of an absorbable material. With the device and procedure of the present invention, there are no external (skin) or internal (mucosal) incisions and the procedure is performed with the central safe zone of the patient's neck.

The surgical time is less than one hour and results in minimal post-surgical pain or discomfort. Recovery is speedy and same day oral diet is achieved. There are no restrictions for post-op CPAP use, when indicted by the medical professional. The procedure can be combined with lingual tonsil ablation (laser, coblation, RF, cautery).

### BRIEF DESCRIPTION OF THE DRAWINGS

Some embodiments of the invention, and of making and using the invention, as well as the best mode contemplated of carrying out the invention, are described in detail below, by way of example, with reference to the accompanying drawings, in which like reference characters designate like elements throughout the several views, and in which:

FIG. 1 is an illustration of the device of the present invention.

FIG. 2 is an illustration of the device of the present invention receiving a suture;

FIG. 3 is an illustration of the suture passer device of the present invention loaded with a suture;

FIG. 4 illustrates alternative positions for the insertion and extraction hooks on the device of the present invention;

FIG. 5A is schematic diagram of the suture passer of the present invention with relative positioning of features;

FIG. 5B is schematic diagram of the suture passer of the present invention with relative positioning of features;

FIG. 6 is another view of the suture passer device of the present invention;

FIG. 7 is the suture passer of the present invention placed next to a barbed suture;

FIG. 8 is the suture passer of the present invention in use on a patient during a medical procedure with robot assisted minimally invasive surgery;

FIG. 9 is a surgical procedure with the present invention viewed on a monitor;

FIG. 10 is robotic forceps grasping and releasing the suture from the suture device during a surgical procedure;

FIG. 11 is an illustration of the robotic forceps reloading the suture onto the suture passer extractor of the present invention;

FIG. 12 is a schematic of a suture loop along the tongue base and around the hyoid after use of suture passer of the present invention with robotic assisted surgery;

FIGS. 13A and 13B illustrate a suture loop along the tongue base and around the hyoid, initiating the suspension process;

FIG. 14A illustrate a suture loop along the tongue base and around the hyoid, indicating partial suspension;

FIG. 14B illustrate a suture loop along the tongue base and around the hyoid, indicating complete suspension;

FIG. 15 is cross sectional view of a patient having robot assisted surgery with the suture passer device of the present invention;

FIG. 16 is a view of the internal surgical area of the patient with the suture passer device of the present invention in first and second positions;

FIG. 17 is an illustration which indicates the safe-zone for a patient to receive the suture passer during surgery;

FIG. 18 is the suture passer of the present invention loaded with a suture and inserted above the hyoid;

FIG. 19 illustrates robotic forceps grasping a suture from the suture passer device of the present invention;

FIG. 20 is an illustration of the suture passer withdrawn empty as the forceps holds the suture;

FIG. 21 is an illustration of the suture passer extracted as the suture is held by forceps;

FIG. 22 is an illustration of the empty suture passer entering the tongue base below the hyoid;

FIG. 23 is an illustration of the suture passer of the present invention reloaded with a suture in the extractor position;

FIG. 24 is cross section of a patient with the suture looped around the tongue base and hyoid in pre-suspension and fixation by the present invention;

FIG. 25 is an internal view of the suture loop of FIG. 24 ready for suspension and fixation;

FIG. 26 is a cross section of the patient with a suture looped around tongue base and hyoid;

FIG. 27 is a cross section of the patient with a suture looped around the tongue base and hyoid to suspend the tongue, hyoid and thyroid;

FIG. 28 is an illustration of the patient with a completed robot assisted minimally invasive suspension of the hyoid.

FIG. 29 is an illustration of a loaded suture passer over the hyoid

FIG. 30 is an illustration of robotic forceps grasping the suture with the suture passer in a second position.

FIG. 31 is an illustration of the suture passer re-loaded with the suture and prepared for extraction.

FIGS. 32 to 36 are implants for use with in the present invention.

### DETAILED DESCRIPTION

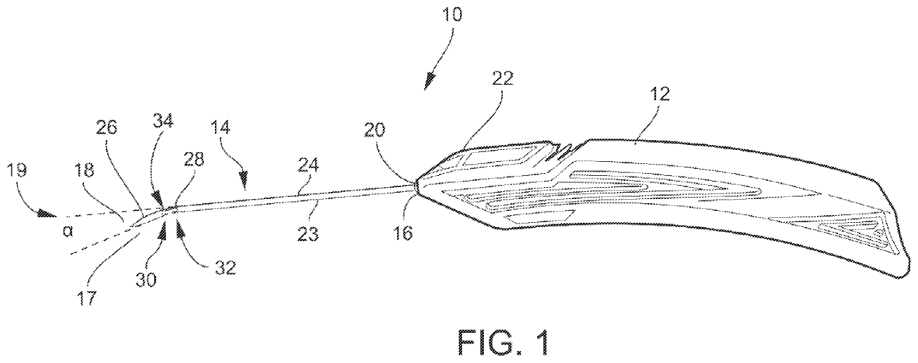

With reference to the Figures, the present invention is herein described. FIG. 1 illustrates the suture passer device 10 of the present invention, which has both inserter and extraction features and capability in a unitary instrument piece. The present invention includes an elongated needle-like passer instrument 14 fastened to a handle grip 12 which receives a first end 16 of the passer instrument 14. A fastener 20 secures the passer instrument 14 to the handle grip 12. A stabilizer piece 22 located inside the handle grip 12 can connect the first end 16 of the instrument 14 by receiving the instrument 16 through the fastener 20. The second end 17 of the passer instrument 14 has a needle tip 18. The solid rigid elongated needle like passer 14 has a smooth surface 24, and preferably a cylindrical shaped body 23, although other shapes which include flat edges are included within the scope. The needlelike passer instrument 14 and tip 18 are of sufficient strength to withstand potential impact with hard tissue, such as bone, or metal instruments when used in surgical procedures. The head section 26 of the passer instrument is positioned at an angle α away from the central axis 19 of the instrument 14. On the surface 24 of the instrument 14, positioned at or in proximity to the head section 26 is a mouth 28 which defines an opening 34 within the body 23 of the instrument 14. The opening 34 is carved or formed out of the instrument 14 and a continuous internal surface 36 which extends from the mouth 28. The opening 34, surface 36 and mouth area 38 create at least one distal hook 30 and at least proximal hook 32. With the device 10 of the present invention, the extractor hook 30 is located at a distal position and used as an extractor for a suture 38 and the insertion hook 32 is located at a proximal position and used as the inserter for a suture 38.

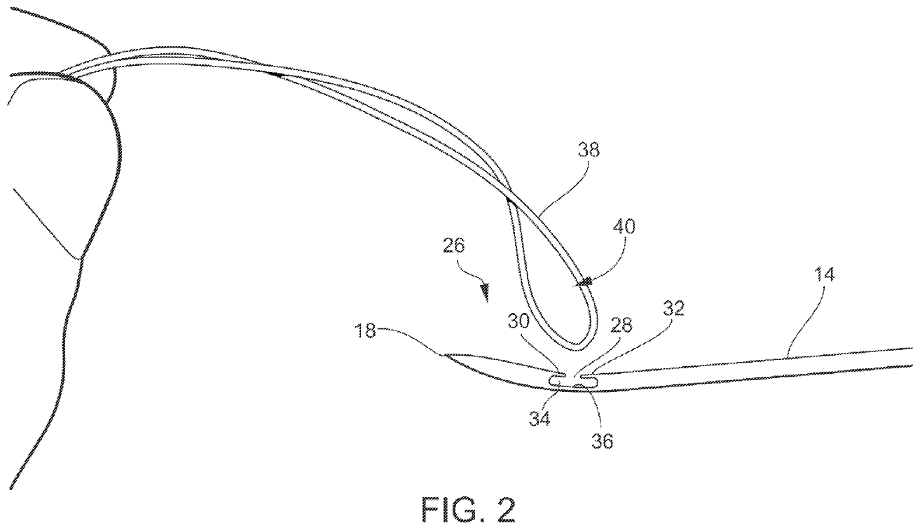

FIG. 2 illustrates a close up of the head section 26, instrument 14 and hooks 30 and 32 of the suture passer device 10 of the present invention. A loop 40 of suture 38 is shown in a figure of eight type shape. The suture 38 is placed into the mouth 28 of the instrument 14 and looped under insertion hook 32 and can be tightened in place by the medical professional. This “loads” the suture passer 10 and it is ready for use in a medical procedure. The suture 38 is inserted into a patient as the suture passer 10 is inserted. After insertion of the suture 38, the suture passer 10 needs to extract the suture 38 in order to form a stitch or loop of suture between an insertion point and an extraction point. For the extraction function of the device 10, the suture 38 is removed from insertion hook 32 and placed under extraction hook 30. By applying a pulling force on the device 10 of the present invention, the suture 38 is pulled by the extraction hook 30 in the same direction with the device 10 and may exit the patient or be brought to a desired position. The insertion hook 32 and extraction hook 30 may be different dimensions or the same, as long as each hook 30 and 32 is capable of receiving and holding the suture 38.

It should be noted that one or both of the hooks 30 and 32 and/or the area near the hooks 30 or 32 can be marked or color coded 42 to visually aid the surgeon in distinguishing an extraction hook 30 from an insertion hook 32 during the surgical procedure.

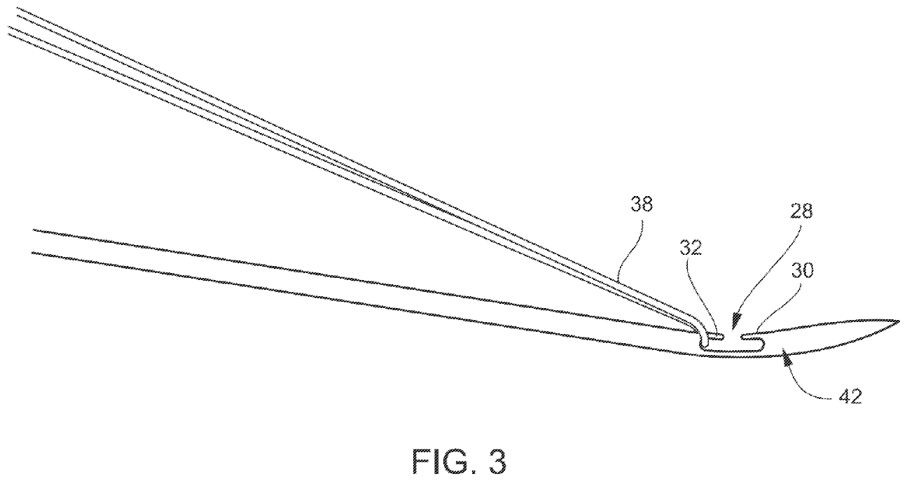

FIG. 3 illustrates a suture passer device of the present invention 10 loaded with a barbed suture. As can be seen in FIG. 3, the suture 38 has been looped around and over proximal hook 32 within mouth 28 and open area 34 of instrument 14 for insertion with the passer 10. The suture 38 has been tightened as indicated by the suture 38 extending in taught manner above instrument 14 and toward a medical professional or fixed point (not shown). In use, the suture passer 10 is inserted into a patient at a desired location and the suture 38 follows the insertion path created by the device 10 as the suture 38 is fixed over hook 32.

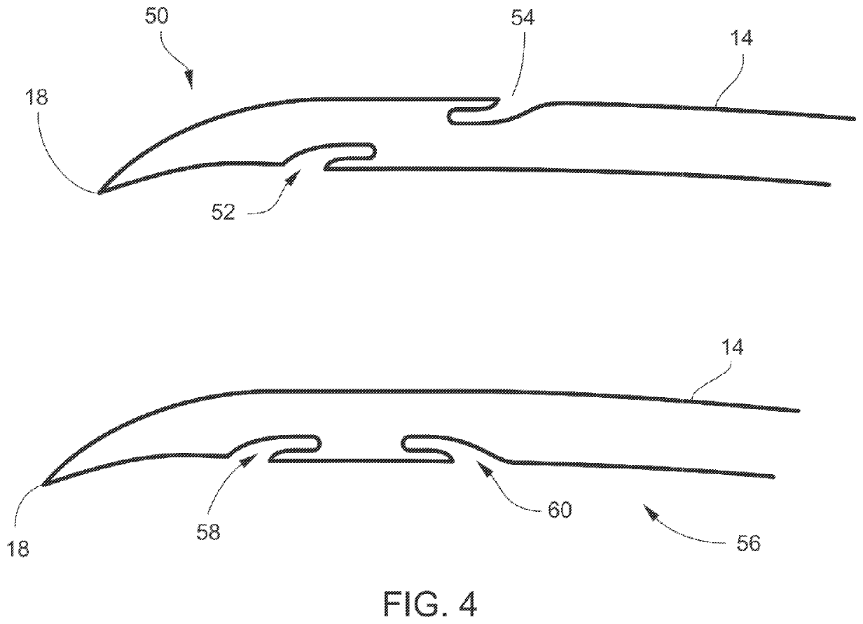

FIG. 4 illustrates alternative embodiments of the present invention which include alternate positions for the hooks along the instrument body 14 to receive a suture 38 on the suture passer 10. The insertion hook may be located at any position along the surface of the instrument 14, including closer to the tip 18 of the instrument than an extraction hook. In like manner, the extraction hook may be positioned anywhere along the surface of the instrument 14. This is shown in FIG. 4, first with reference to alternative embodiment 50, which includes the insertion hook 52 located closer to tip 18 of instrument 14 than extraction hook 54. Further, the extraction hook 54 may be positioned on opposite side of instrument 14 from the insertion hook 52. Those of skill in the art recognize that position of insertion hook 52 and extraction hook 54 may be switched in positions and such an embodiment is within the scope of the invention. In alternative embodiment 56, the insertion hook 58 and extraction hook 60 are located on the same side of the instrument 14. The insertion hook 58 is located closer to tip 18 than extraction hook 60 here as well. In these embodiments, the suture 38 may be placed onto the suture passer 10 in similar manner as described herein.

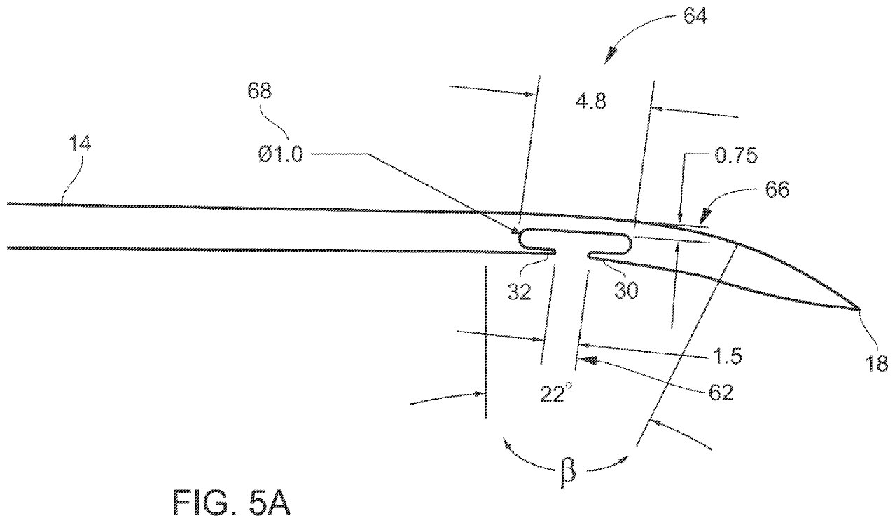

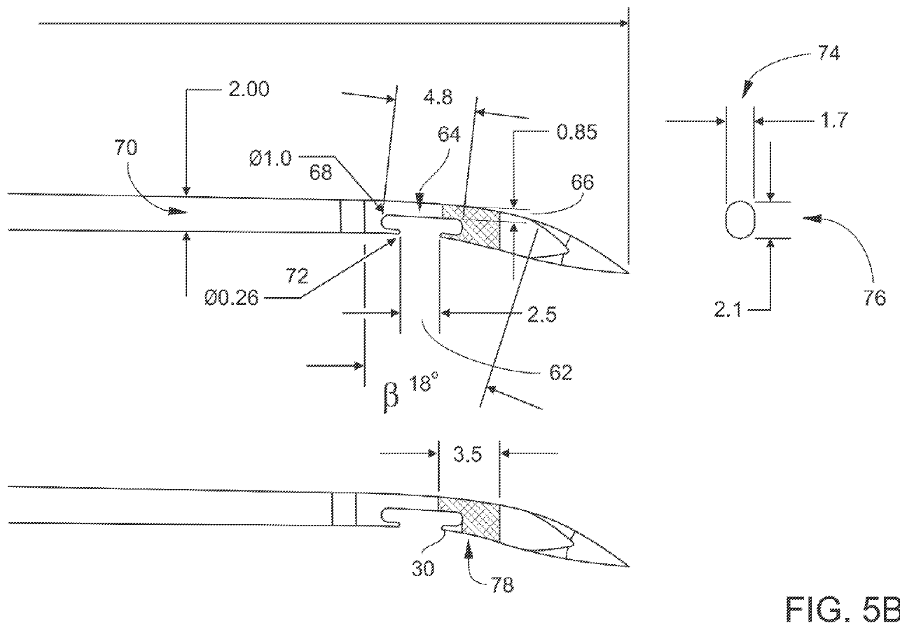

FIG. 5A is a schematic to illustrate various non-limiting dimensions of an example of the present invention. In FIG. 5A, there is shown an instrument 14 of the suture passer 10 formed with known suitable materials for suture passers, such as metals, alloys, polymers, composites, etc., and by methods known in the art. The mouth opening 28 of the instrument 14 has a length 62 and is 1.5 millimeters in this example, although a range of approximately 0.5 to 4 millimeters is within the scope. The internal opening 34 formed by the extraction hook 30, insertion hook 32, and the internal surface 36 has a length 64 and is shown as 4.8 millimeters in FIG. 5A. The internal opening 34 and internal surface 36 provide a remaining thickness 66 of approximately 0.75 millimeters to external surface of the instrument 14 from internal surface 36. The diameter 68 is indicated as [symbol] 1.0. An angle β from the body 23 of instrument 14 to head 26 of instrument is also shown, and in this particular example, the angle is 22 degrees.

A second non-limiting example is shown in FIG. 5B, where there is again shown an instrument 14 of the suture passer 10 formed with known suitable materials for suture passers, such as metals, alloys, polymers, composites, etc., and by methods known in the art. The mouth opening 28 of the instrument 14 has a length 62 and is 2.5 millimeters in this example. The internal opening 34 formed by the extraction hook 30, insertion hook 32, and the internal surface 36 has a length 64 and is shown as 4.8 millimeters in FIG. 5B. The internal opening 34 and internal surface 36 provide a remaining thickness 66 of approximately 0.85 millimeters to external surface of the instrument 14 from internal surface 36. The diameter 68 is indicated as [symbol] 1.0. An angle β from the body 23 of instrument 14 to head 26 of instrument is also shown, and in this particular example, the angle is 18 degrees. A body thickness 70 is shown as 2.0 millimeters. The insertion hook 32 in FIG. 5B has a diameter 72 of 0.26 millimeters. The instrument in this example has an oval shape and there is also provided a width 74 of 1.7 millimeters and height 76 of 2.1 millimeters. A visually marked area 78 near extraction hook 30 is shown as 3.5 millimeters in this non-limiting example.

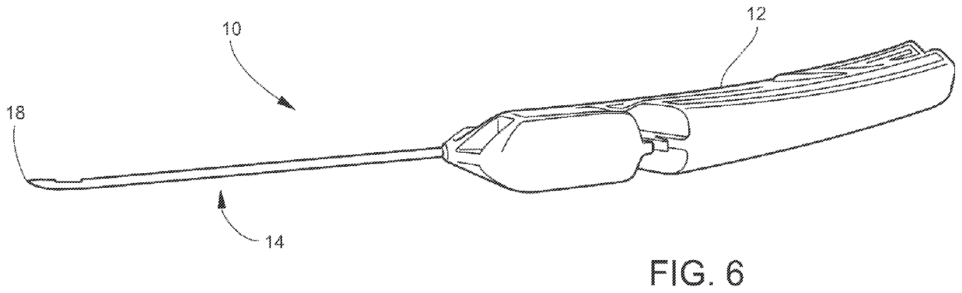

FIG. 6 is another view of the suture passer device 10 of the present invention. The handle grip 12 may be made of materials and methods known in the art and may include grooves, ridges, or other means for aiding a medical professional to hold the device 10. The handle 12 length similarly may be of any sufficient size to allow the medical profession to grasp the overall device 10 and have ease of maneuverability with the device during procedures. The handle 12 includes an opening or similar means at an end for receiving the instrument 14 and is fastened by fastening means and methods known in the art. An internal stabilizing piece 22 is also used for receiving end of the instrument 14 as well as support. In an embodiment, the handle 12 is an integrated part of the instrument 14, and without the need for separate fasteners or separate instrument and handle sections.

The overall length of the instrument 14 from the handle 12 to the tip 18 may be a range of approximately 6 centimeters to 12 centimeters, with 9 centimeters as fitting for surgical procedures within the scope herein. The instrument 14 is of sufficient length to enter a patient's neck and penetrate tissue past the hyoid bone and tongue base and into the patient's throat cavity and airway area during a surgical procedure. In this manner the suture passer with a loaded suture can be visible to a camera attached to a robotic arm and flex system which is inserted into a patient's mouth during surgery. The images from the camera are sent to a display for the medical professional's live viewing during surgery.

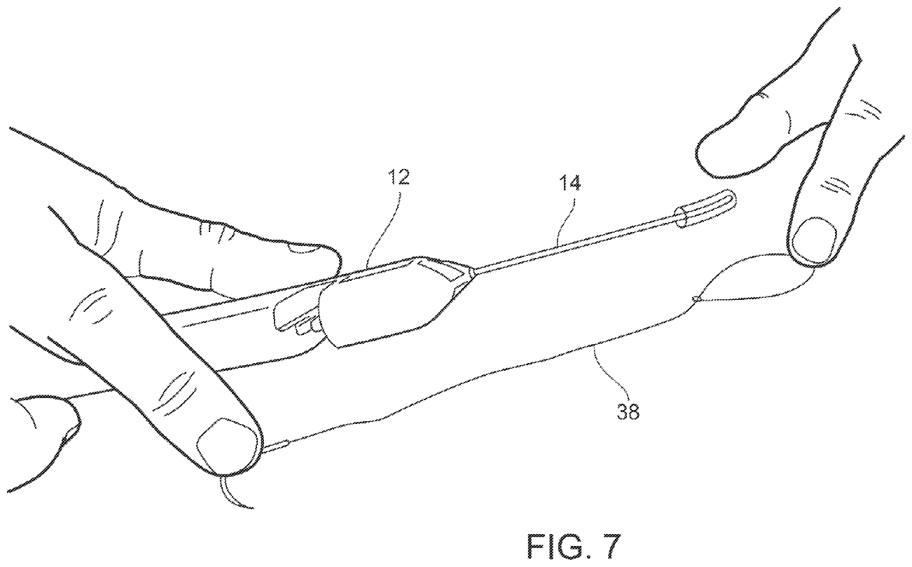

FIG. 7 illustrates the suture passer 10 of the present invention adjacent to a barbed suture. A No. 2 barbed suture is suitable for the device of the present invention, although other sutures known in the art are suitable for use with the suture passer device 10. The suture 38 is of sufficient length to extend with the suture passer into a desired surgical area

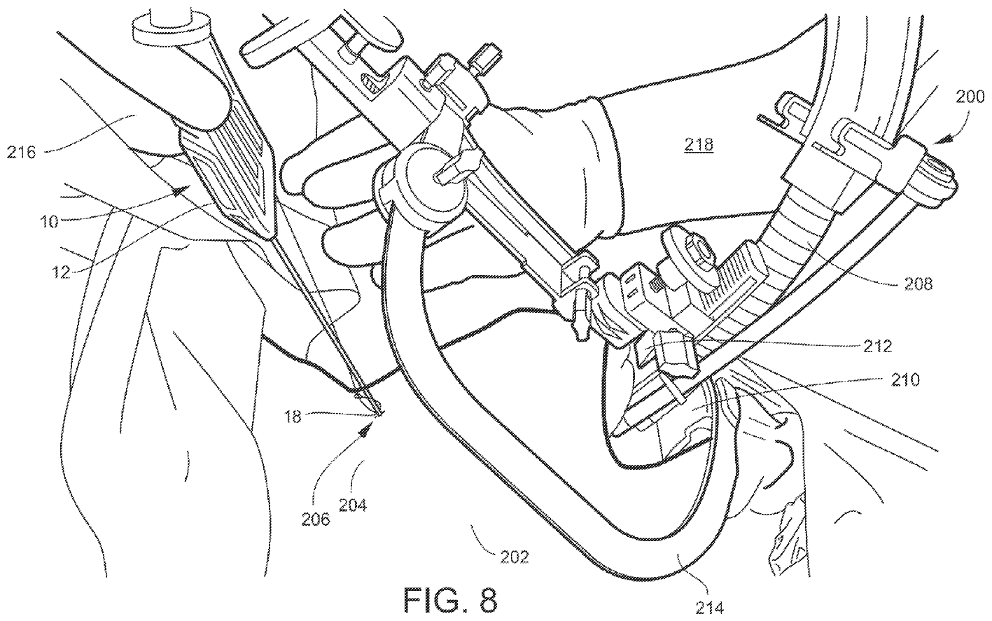

Referring to FIG. 8 there is shown the suture passer device of the present invention 10 in use with patient 202 receiving the medical procedure 200. The suture passer 10 is shown held by a medical professional 216 at its handle 12 as the needle tip 18 of the device 10 is inserted at the midline 206 of the neck 204 of the patient 202. The patient 202 during this procedure of robotic surgery has robot arm 208 inserted through his mouth while clamp 214 holds the patient's mouth in open position and a tongue blade 212 is used to position the tongue for the surgery. The suture passer 10 enters the neck 204 at the midline 206 just under the location of the hyoid to deliver a barbed suture attached to the suture passer at the insertion hook.

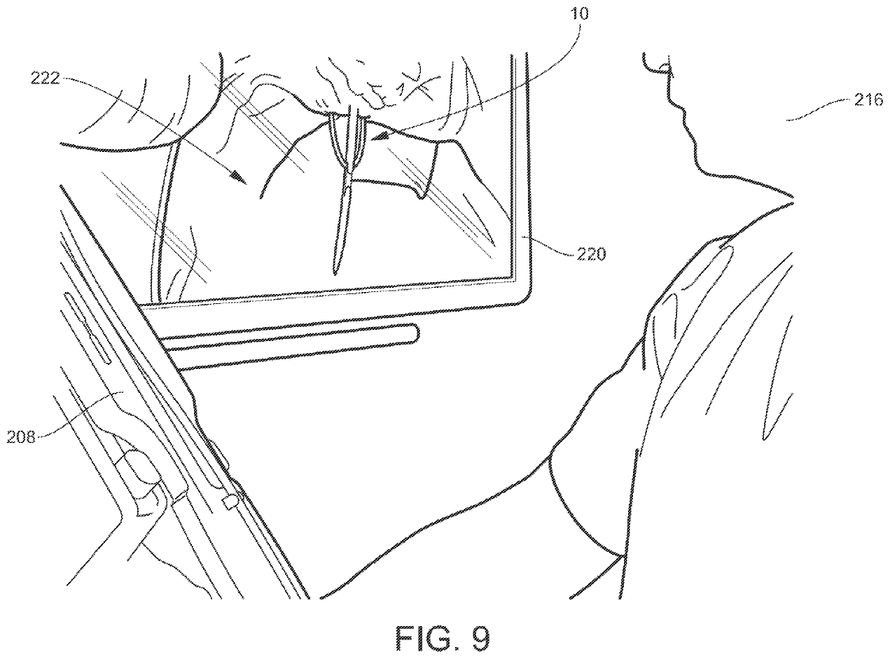

Referring now to FIG. 9 there is illustrated a television monitor 220 which provides the image from inside the patient from cameras attached to the robot 208 which have been inserted into the patient's mouth and throat. A medical professional 216 can view the images of the suture passer device 10 entering the tongue base/vallecula. The suture passer 10 is loaded with barbed suture 38 and brought to a position far enough away from the tongue base so that medical professional 216 may begin work with the robotic instrument and have visibility in the surgical area inside the mouth of the patient 222. This is the insertion step of the process with the suture passer in a first position.

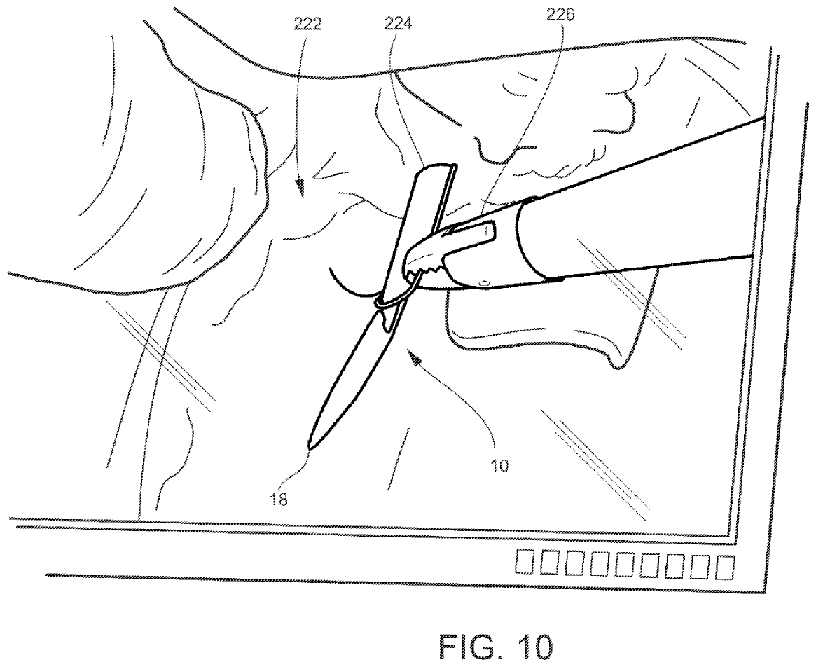

As seen in FIG. 10 the inside of the mouth and surgical area of patient 222, there is shown forceps grasping and releasing the barbed suture 38 which has been fastened to the suture passer 10. The suture passer 10 is shown protruding from the first opening 224 created when tip 18 of the suture passer 10 pierced the tissue. The suture passer 10 and its elongated body 14 extend through the tissue with suture 38 fastened to insertion hook 32. During this time the tongue blade remains in position as it exposes the center compartment of the tongue base.

Referring now to FIG. 11 there is shown again the medical professional 216 viewing the monitor 220 and using the forceps 226 to reload the barbed suture 38 onto the suture passer 10 at the extractor hook 30.

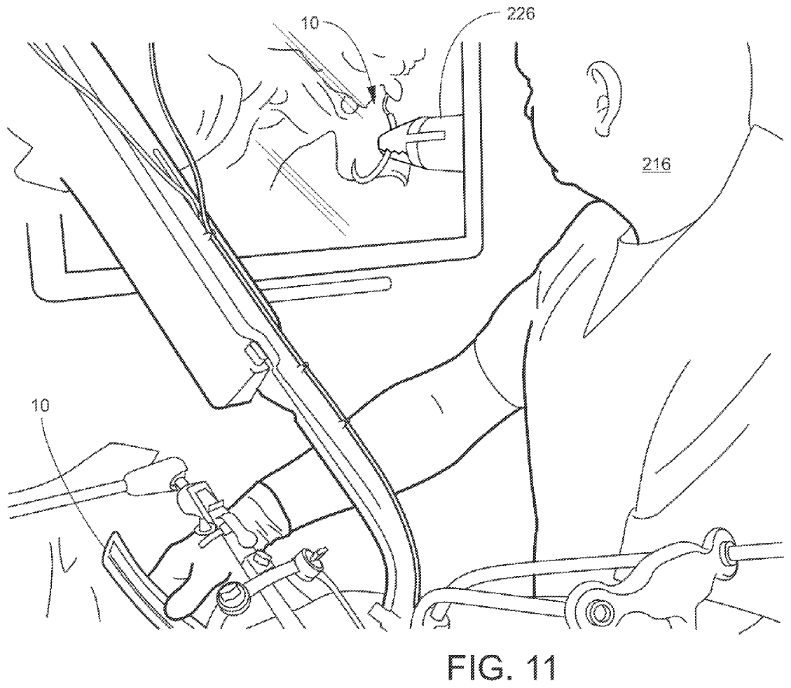

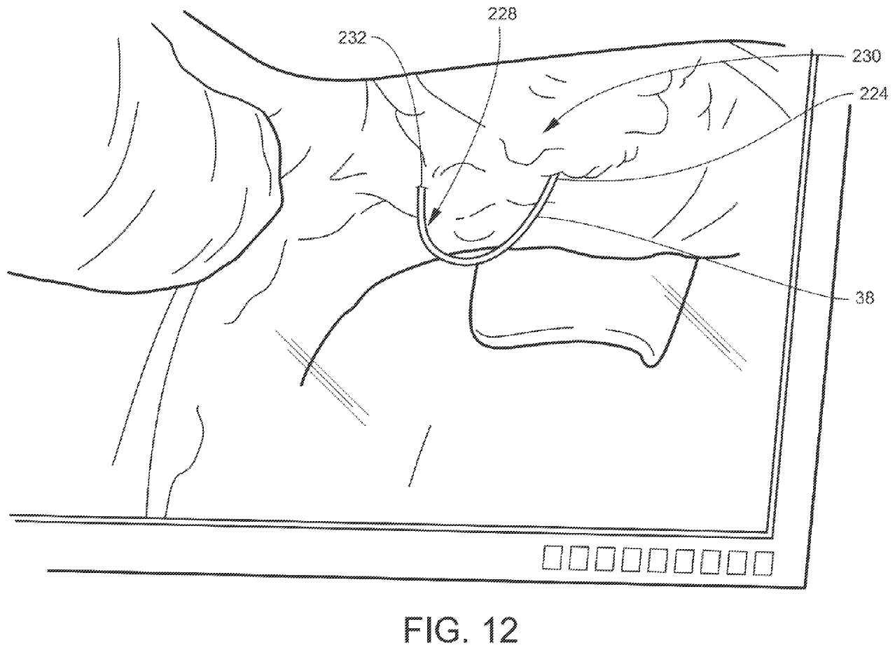

In FIG. 12, the device of the present invention 10 has been extracted through a second position. As can be seen, the suture 38 forms a suture loop 228 from entering insertion opening 224 and returning through extraction opening 232. The suture loop 228 loops along the tongue base and around the hyoid before suspension is performed.

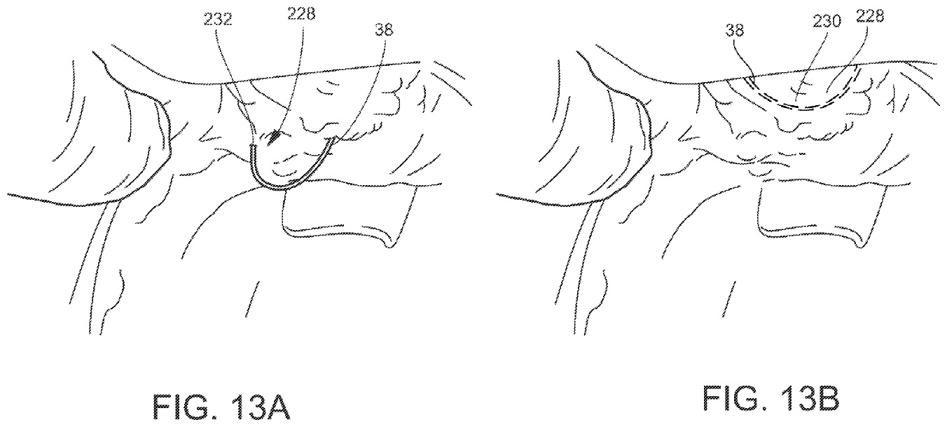

Referring to FIGS. 13A and 13B there is shown the suture loop 228 along the tongue base and around the hyoid initiating suspension. The suture loop 228 is pulled upward through insertion opening 224 and extraction opening 232. As shown in FIG. 13B the suture loop 228 pulls the tissue upward enlarging the cavity opening inside the patient. This will assist in providing a clear respiratory path for a patient who suffers sleep apnea.

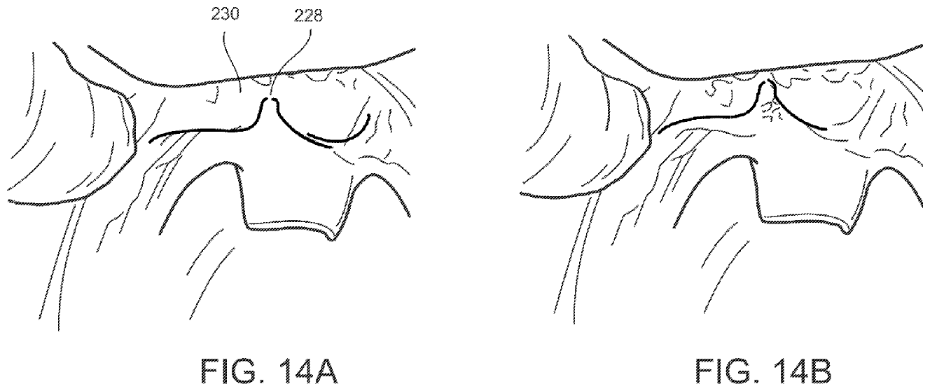

Referring to FIGS. 14A and 14B there is shown the suture loop 228 along the tongue base and around the hyoid 250. In FIG. 14A there is shown partial suspension of the tissue. In FIG. 14B there is shown complete suspension.

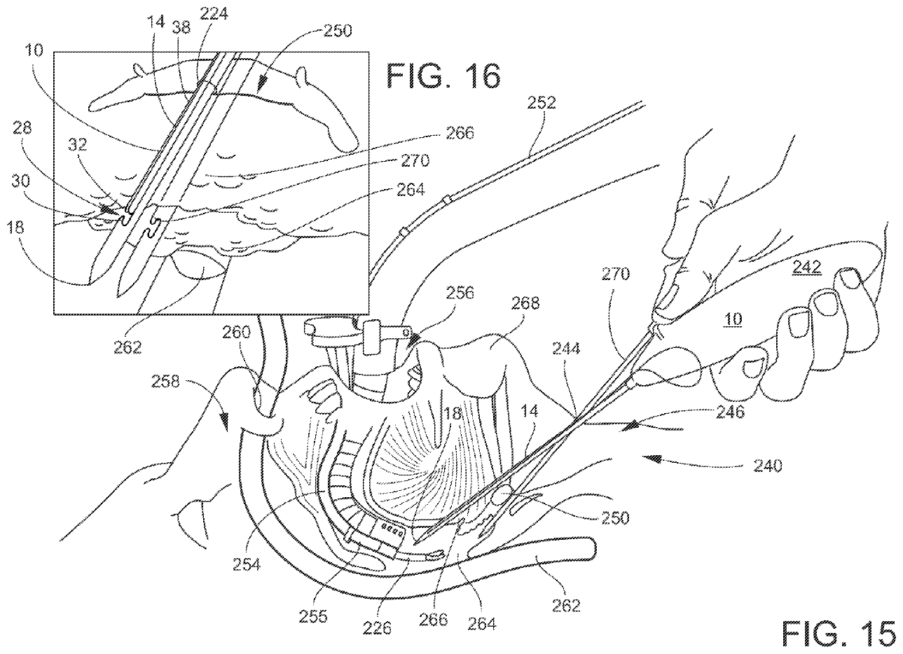

Referring to FIG. 15 there is shown a cross-section of a patient 240. This patient 240 is undergoing a robotic surgery with the device of the present invention 10. Nasal tracheal intubation 262 is inserted through nostril 260 of patient's nose 258. The robot arm 252 has been inserted through the patient's mouth 256 with the robotic flex system 254 extending into the patient's throat. The robotic flex system includes a camera with light 255 for visibility in the area of the medical procedure. Forceps may be attached with the flex system as well. Within FIG. 15 there is shown hyoid bone 250, epiglottis 264, and lingual tonsil 266 as part of tongue base. Mouth 256 and mandible 268 are maintained in position during surgery by clamp 214 (FIG. 8). As shown in FIG. 15 the suture passer of the present invention 10 is inserted at a single entry point 244 from a first position 242 so that tip 18 and elongated body 14 of the suture passer 10 pass over the hyoid bone 250. In this manner tip 18 of suture passer having barbed suture 38 loaded onto insertion hook will pass over the hyoid bone and through tongue base into throat cavity of patient 240. The forceps of flex system and robotic arm 252 at this point can grasp the suture 38 from the suture passer 10 and release the suture 38 from the suture passer 10. Since the suture 38 has to make a loop, the entry and exit of the suture 38 (around the hyoid) needs to be in different locations. To achieve this, the suture passer 10 is moved to a second position 270 without removing the suture passer through the entry point 244. The curved tip and angled head section 26 at second end 17 of the instrument 14 (See, FIGS. 1 and 2) facilitates this action as the surgeon can rotate the handle 12 and puncture the tongue tissue in a second location for exit of the suture 38 which is different than the initial entry location of the suture 38. The suture passer 10 in second position 270 will pass below the hyoid bone 250 and tip 18 will extend into patient's throat cavity n the surgical area. The suture 38 which has been held by forceps of flex system 254 will now be loaded onto the extractor hook 30 of the suture passer 10. In this manner the suture 38 has entered at a first position and exits at a second position to loop around the hyoid bone. Those of skill in the art will note that position one 242 of device 10 and position two 270 may be done in either order.

Referring to FIG. 16 there is shown the internal view of FIG. 15. In this Figure, suture passer of device 10 enters through insertion opening 224 with suture 38 loaded onto insertion hook 32 within mouth 28 of the suture passer. The suture passer 10 as can be seen from insertion opening 224 passes over hyoid bone 250 and over lingual tonsil area. The tip 18 extends past epiglottis. In this manner the device of the present invention 10 and elongated body 14 extend through insertion opening 224 with suture 38 to a sufficient distance allowing medical professional increased visibility in surgical area. The suture 38 is unloaded from suture passer 10 by robotic forceps. The suture passer 10 is then moved to a second position 270 behind hyoid bone 250 and behind lingual tonsil 266. The empty suture passer 10 in position two may now receive suture 38 into extraction hook 30. As can be seen in FIG. 16 nasal tracheal intubation 262 remains in place for patient.

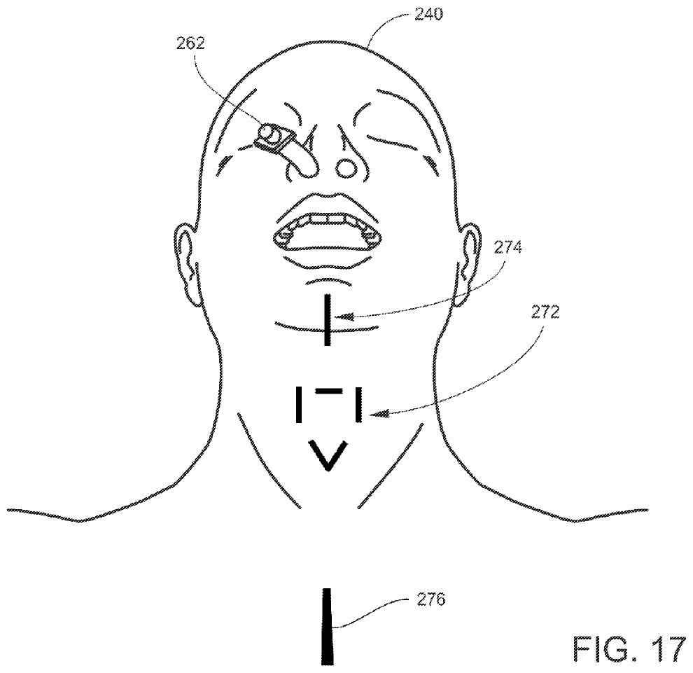

FIG. 17 is an illustration of patient 240 receiving nasal tracheal intubation 262 and in preparation for robotic surgical procedures in use of the device of present invention 10. Patient 240 has safe zone 272 located along the anatomical midline landmarks for the chin 274 and sternal notch 276. The safe zone 272 is located in patient's throat below chin and is the location for the suture passer device of the present invention to enter the patient's body.

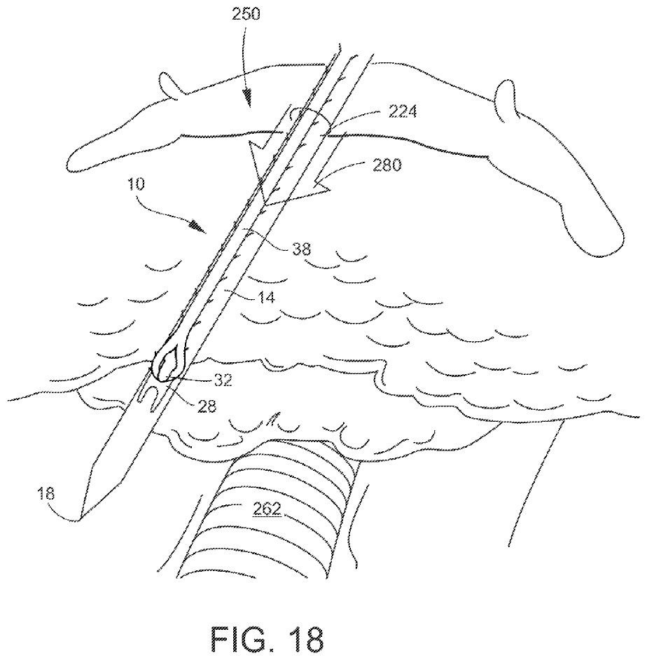

As the suture passer 10 is inserted into safe zone 272 it will be inserted above the hyoid bone 250. This is shown in FIG. 18 where the suture passer 10 acting as inserter and loaded with barbed suture 38 penetrates tissue creating insertion opening 224 as the suture passer 10 is pressed downward as indicated by insertion movement demonstrated by insertion arrow 280. The suture 38 has been loaded around and under insertion hook 32 previously by the medical professional so that suture 38 received in mouth 28 and under hook 32, so that the suture 38 may pass down into the patient and over the hyoid bone 250 and extend with the suture passer into the throat cavity of the patient. Intubation tube 262 is also shown in FIG. 18 indicating the close proximity of the surgical area.

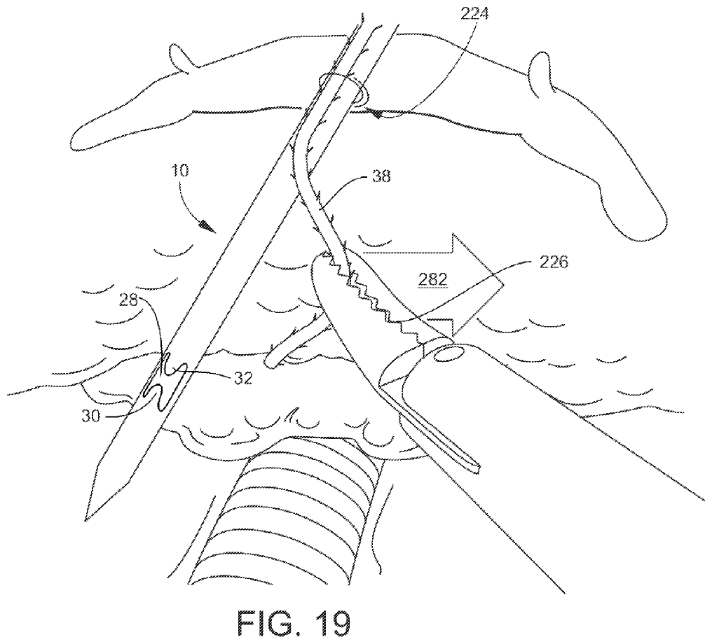

In FIG. 19, the robotic arm with robotic forceps 226 is shown with the suture passer device 10 in the surgical area from FIG. 18. The robotic forceps 226 grasps and releases the barbed suture 38 from the suture passer 10 and the insertion hook 32 over the mouth 28 along the body of the instrument 14. The robotic forceps 226 moves the suture 38 away from the suture passer 10 as indicated by arrow 282.

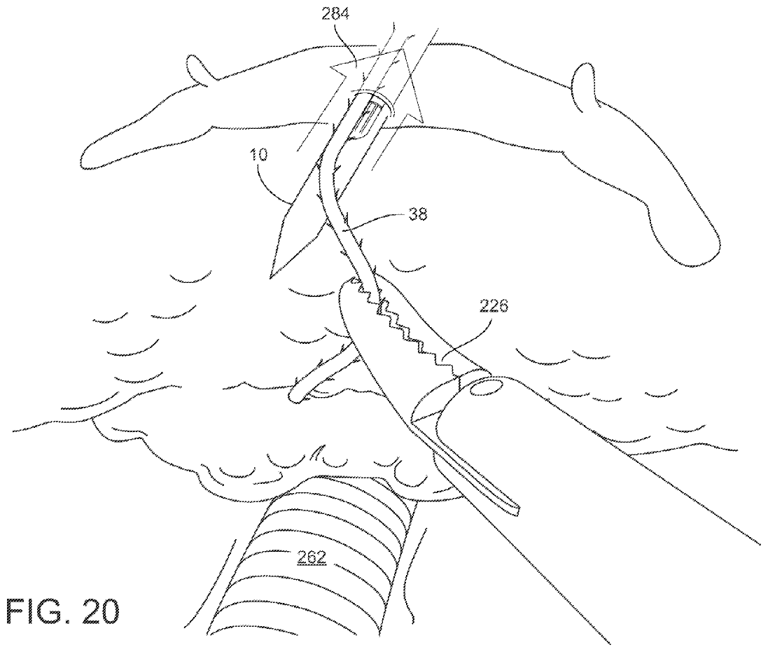

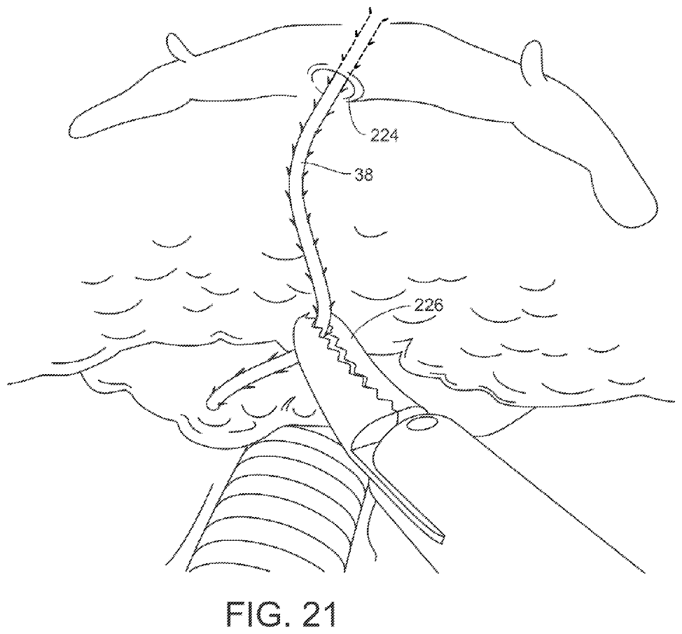

Continuing the procedure in FIG. 20, as the forceps 226 holds the suture 38 the suture passer 10 of the present invention is withdrawn empty back through insertion opening 224. This is indicated by direction arrow 284 in FIG. 20. With FIG. 21, the suture passer 10 of the present device has been fully extracted and the forceps 226 remains holding the suture 38.

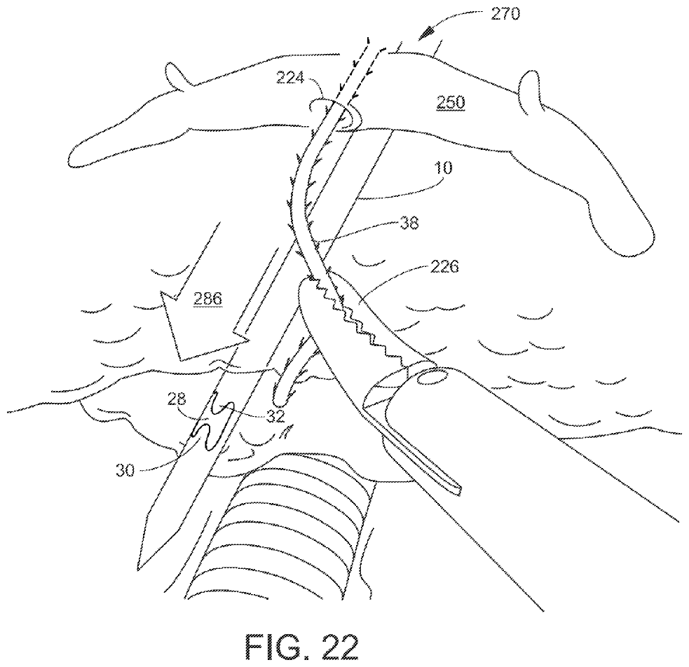

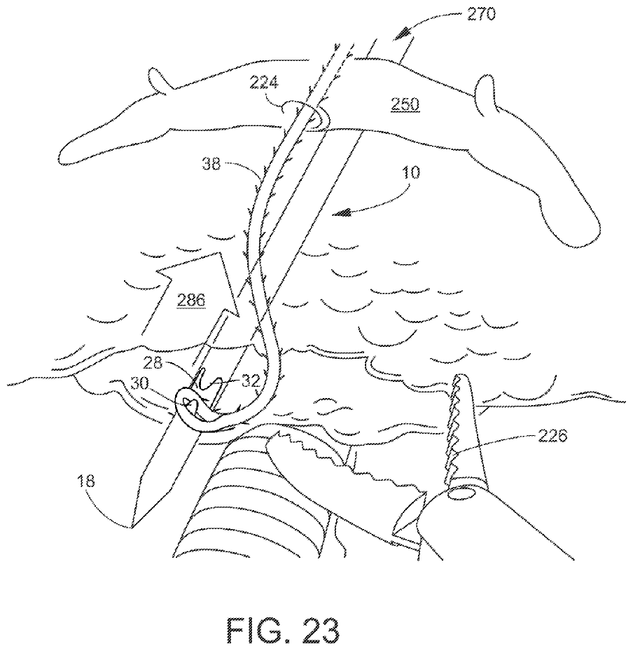

In FIG. 22, the suture passer (empty) enters the tongue base below the hyoid 250 in a second position 270. The forceps 226 is shown still grasping the suture 38, which is extending from insertion opening 224. As the suture passer 10 moves downward as indicated by direction arrow 286, the mouth 28 and hooks 30 and 32 on the instrument 14 become visible to the surgeon by the robotic camera.

The medical professional reloads the suture 38 with the forceps into the mouth 28 located on suture passer 10. This time, the suture is loaded onto extraction hook 30 which is located closer to the tip 18 in this embodiment. Once the barbed suture 38 has been reloaded onto the suture passer in the extraction position, the suture passer 10 may begin withdrawing and extracting from the surgical area and patient. This is indicated by withdrawal arrow 286. The forceps 226 are shown in open position after the suture 38 has been fastened to the suture passer.

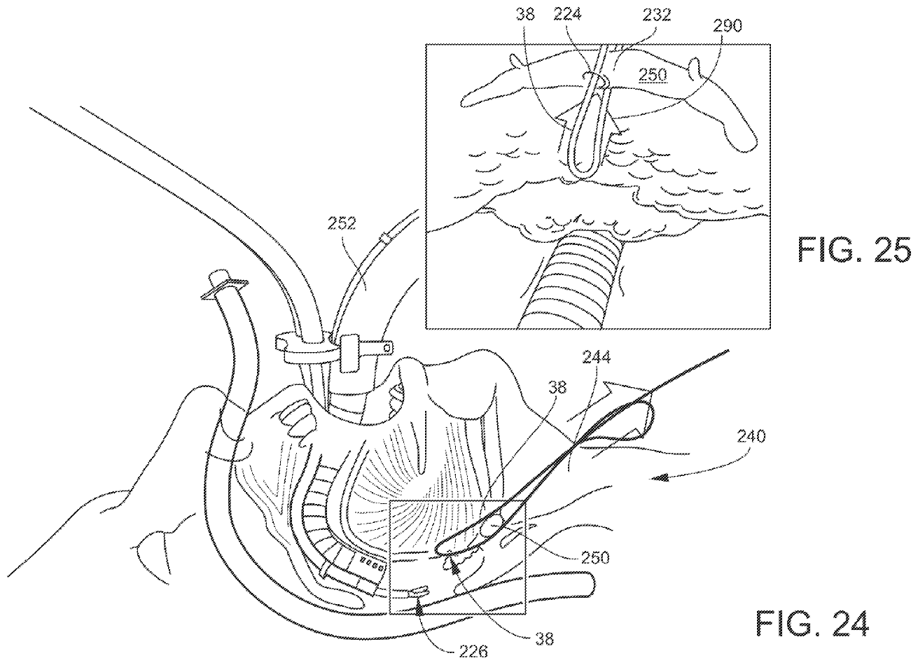

FIG. 24 is a cross-section of the patient 240 with the suture 38 looped around the tongue base and hyoid 250, which are now ready for suspension and fixation. As shown in FIG. 24 the suture 38 enters and exits the patient from the single entry point 244.

FIG. 25 is an internal view of the suture loop from FIG. 24 which is ready for suspension and fixation. The suture 38 is shown passing through first opening 224 on one side of hyoid bone 250 and into second opening 232 on opposite side of hyoid 250. The suture 38 is then pulled upward by medical professional as indicated by arrow 290.

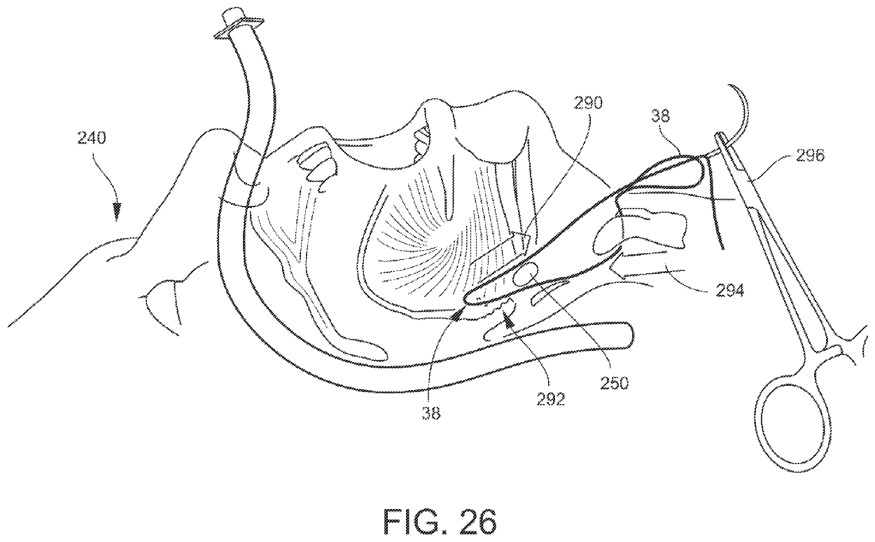

FIG. 26 is a cross-section of the patient 240 with the suture 38 looped around tongue base 292 and hyoid 250 which are now ready for suspension. A needle is also used for fixation of barbed suture 38 on the thyroid cartilage 294. A medical instrument 296 external to the patient's body such as a clamp, may grasp the suture ends 38 which now protrude from the patient 240 and pull the suture upward for tissue suspension as indicated by arrow 280.

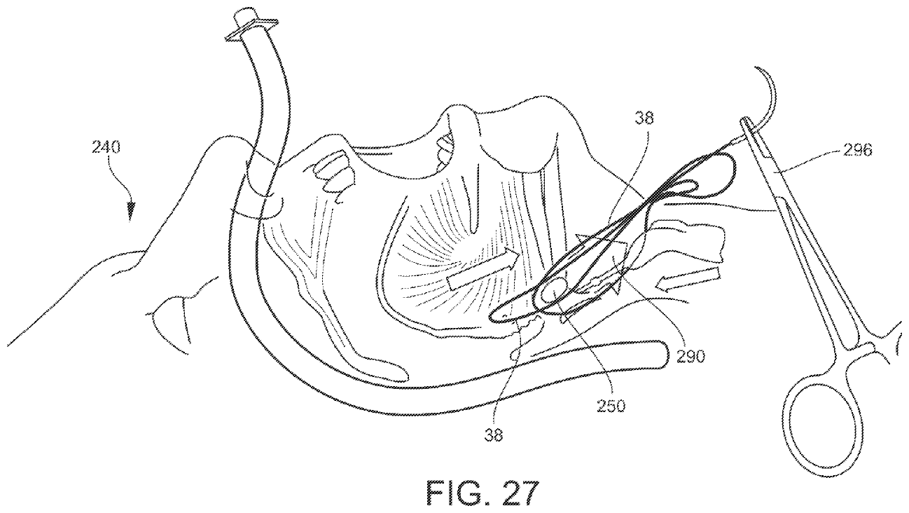

In FIG. 27, the cross-section of the patient 240 illustrates the barbed suture 38 around tongue base and hyoid, thereby suspending the tongue, hyoid and thyroid and opening the airway of the patient. When the medical professional pulls and tightens the suture 38 in a direction away from patient (noted as arrow 290), fixation is done. This is on the thyroid cartilage.

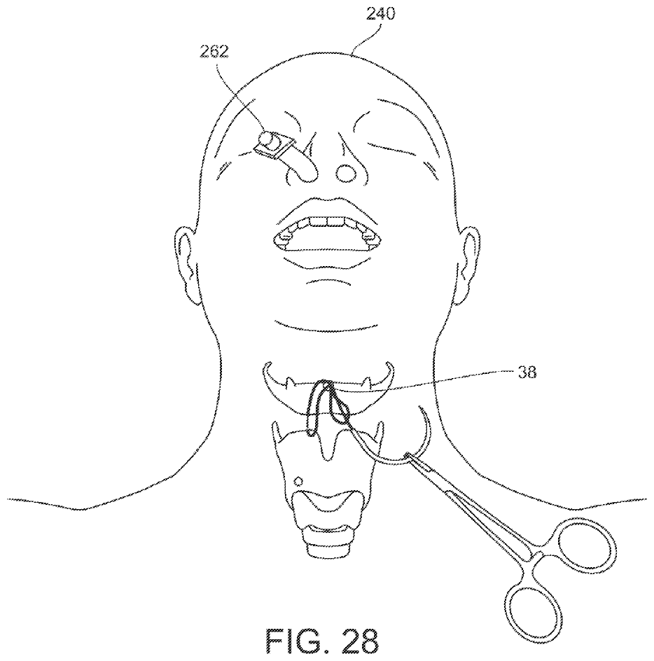

In FIG. 28, there is illustrated the patient 240 with the suture 38 fixed in position and tied. This is the completion of the robot-assisted minimally invasive suspension of the hyoid and tongue base so as to improve and increase a patient's airway to treat OSA.

The device and procedure of the present invention provides a minimally invasive robotic surgery for treatment of OSA which includes the suspension of the hyoid and tongue base to open the patient's airway. There are no external or internal incisions made during the procedure and the barbed sutures used are absorbable. The procedure is robotic-assisted and thereby provides an increase in visualization for the surgeon, as well as safety and accuracy of the procedure. Further, there are no permanent implants in the patient and any OSA patient with tongue collapse may benefit from the procedure. There is no AHI cut off limit and there is no need for pre-operation DISE. The procedure may be combined with other airway procedures. The use of the device and procedure has indicated that there are no adverse events or complications.

In addition to the devices and procedures described above, the suture passer can be used in minimally invasive suspension of the hyoid, with and without robotic assistance, with epiglottoplasty. The purpose is to secure or lift and suspend the epiglottis to open the airway of the patient.

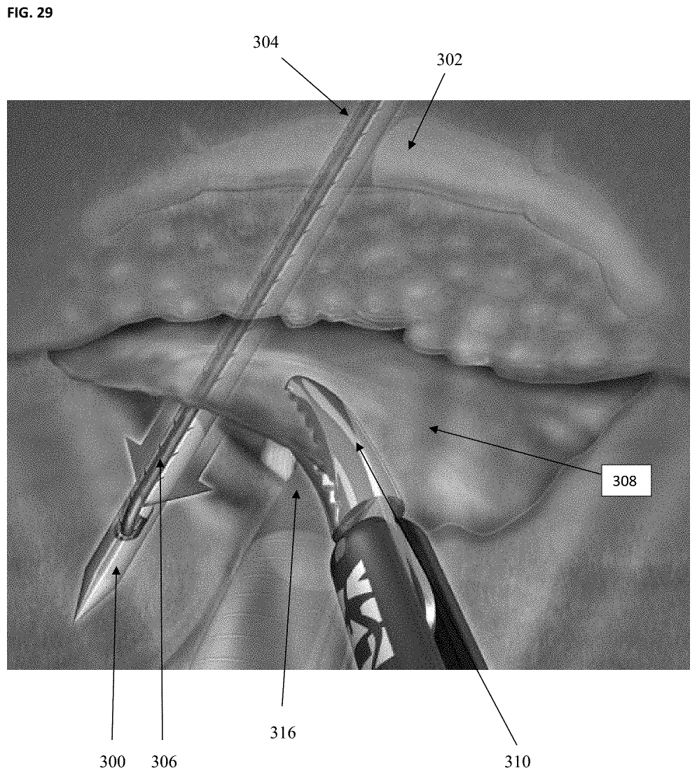

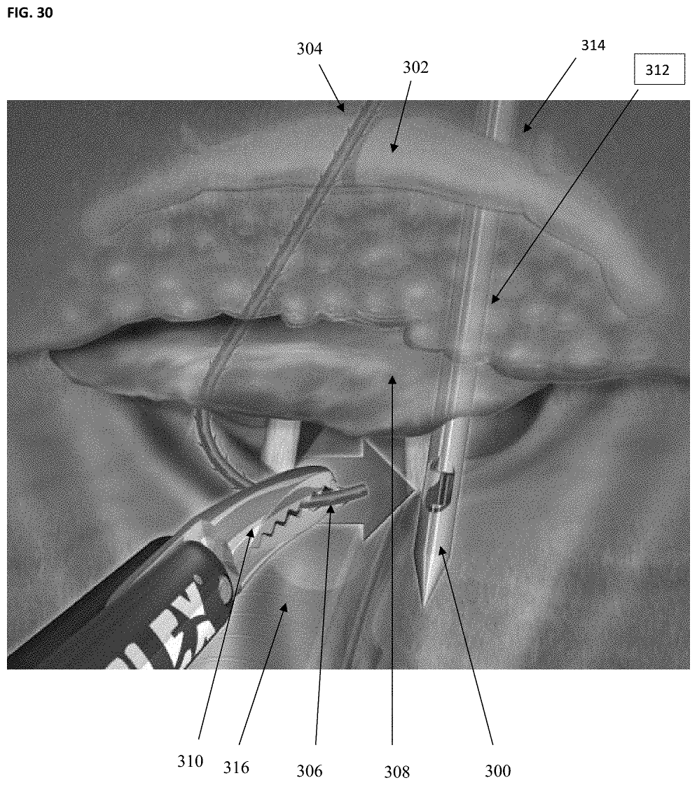

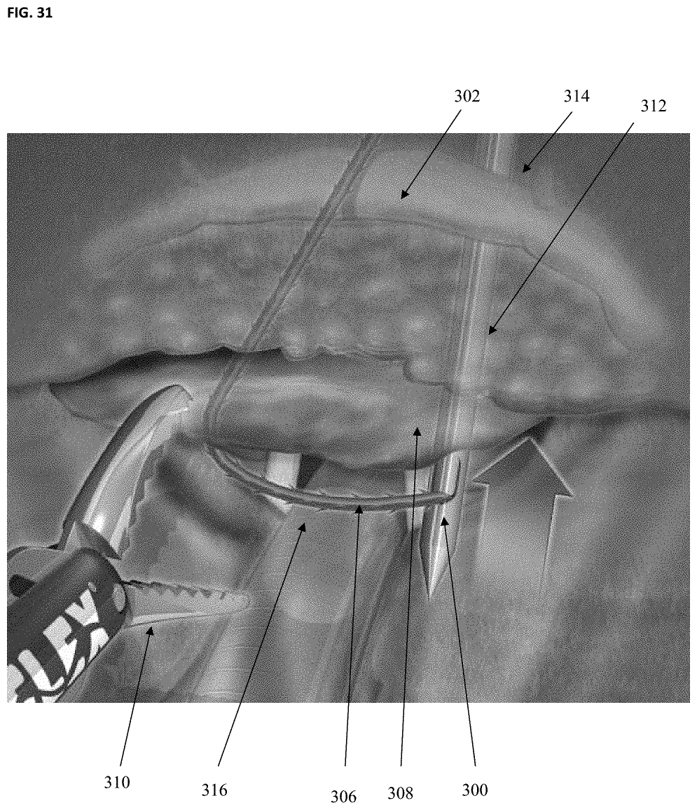

FIGS. 29 through 31 demonstrate the procedure of the present invention with a suture passer device 300 passing over the hyoid bone 302 in a first position 304 with a loaded barbed suture 306, with extension of the device beyond the epiglottis 308. The suture 306 is then removed from the suture passer 300 and held by robotic forceps 310 while the suture passer device 300 is moved to a second position 312, behind or on opposite side of the hyoid bone 302 from the first position 304. The suture passer device is then reloaded by the forceps 310 and the suture passer device 300 is extracted with the barbed suture 306. In this manner, the suture 306 loops around the epiglottis 308 to lift and suspend the epiglottis 308 and thereby open the air passage way 316 where the breathing tube is located during a procedure. The suture passer device for the procedure may be those described previously herein or an alternative slotted device as shown in FIGS. 29-31.

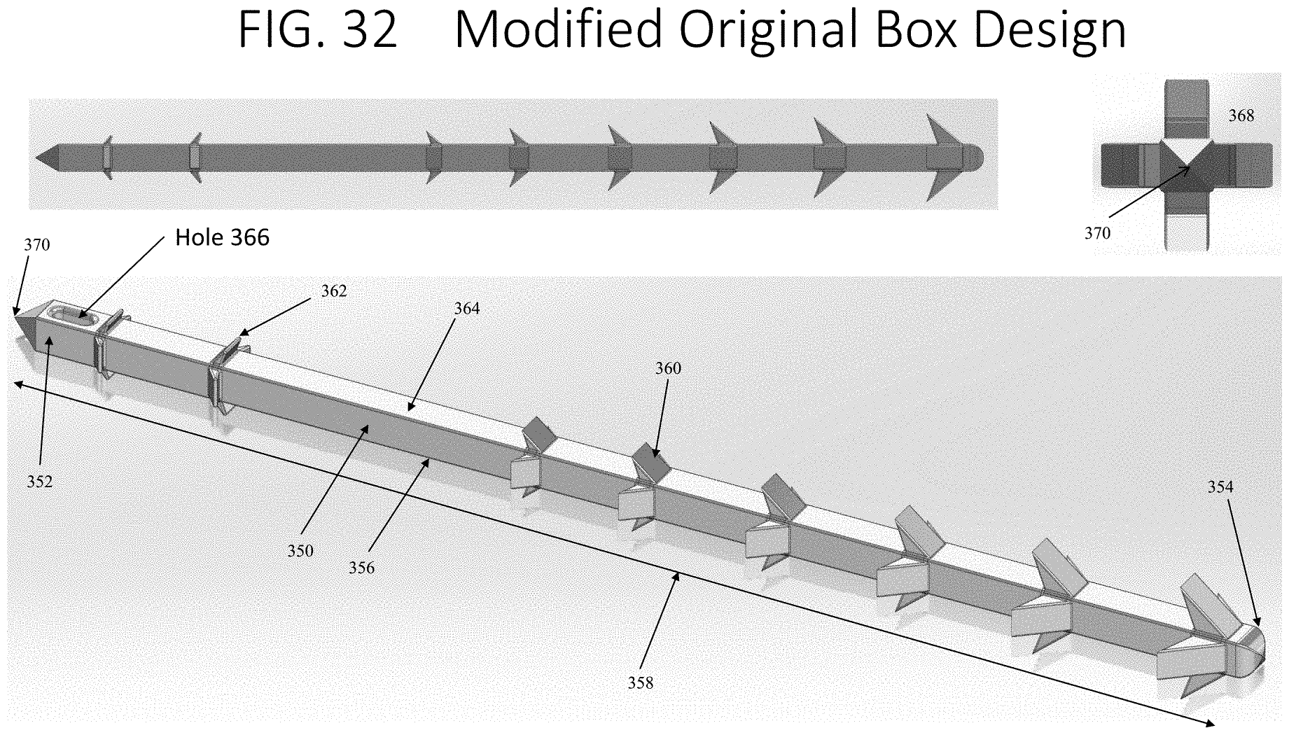

Barbed sutures and bioabsorbable implants for epiglottoplasty may be used. Implant designs such as those illustrated in FIGS. 32 through 36 are particularly suitable for the present invention.

FIG. 32 shows a barbed implant 350, which has a first end 352 and a second end 354 between an elongated body 356 having a length 358 of approximately 35 mm, and a range of 25 mm to 50 mm is within the scope, but other lengths are within the scope of the invention. A pointed tip 370 is located at the first end 352 of the barbed implant 350. A plurality of back facing barbs 360 are aligned along the body 356 of the implant 350, with six shown in FIG. 32. There are also one or more front facing barbs 362 positioned along the elongated body 356, with two front facing barbs shown. Between the front facing barbs 362 and the back facing barbs 360 is a barbless area 364. Positioned at or near the first end 352 of the implant 350 is a hole for receiving sutures. An axial view of the box design 368 is also included by looking at the pointed tip 370 of the first end 352.

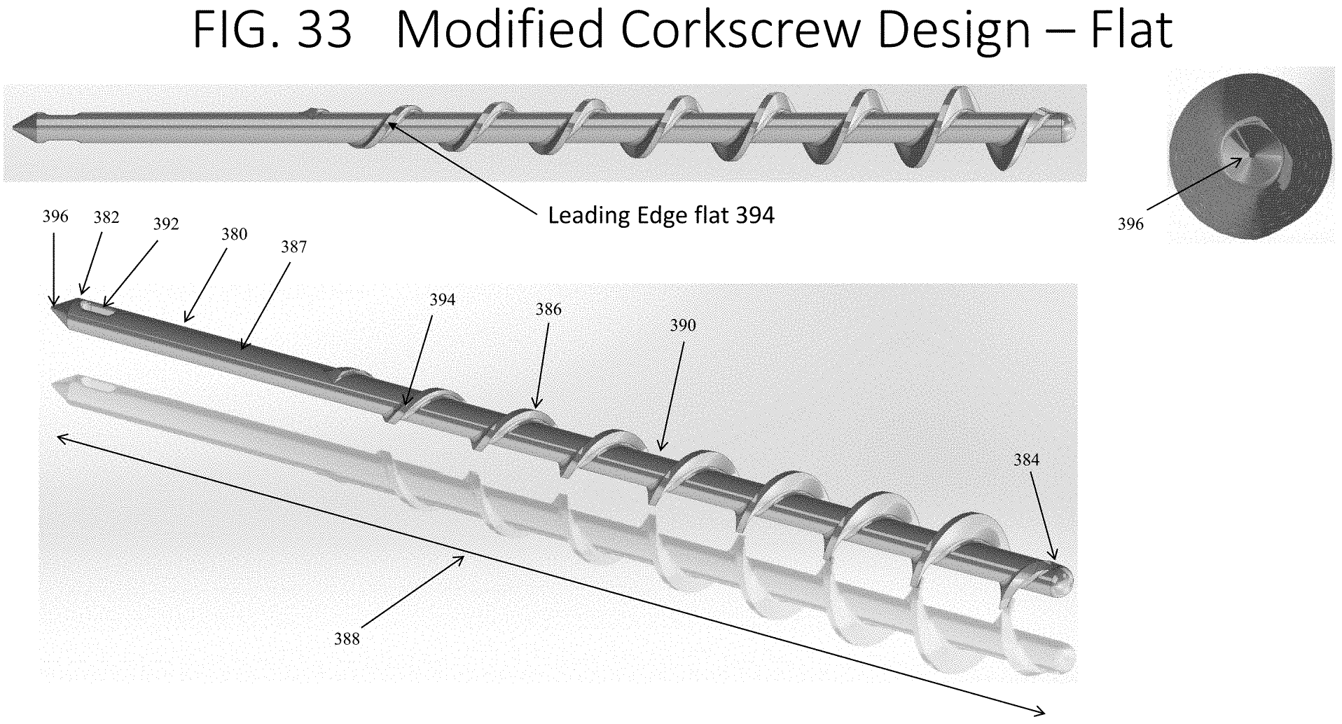

FIG. 33 shows an implant 380, which has a first end 382 and a second end 384 between an elongated rod like body 387 having a length 388 of approximately 35 mm, and a range of 25 mm to 50 mm is within the scope, but other lengths are within the scope of the invention. A pointed tip 396 is located at the first end 382 of the barbed implant 380. A corkscrew 386 runs the length 388 of the implant 380 along its surface 390, with the number of corkscrew turns being eight turns, although other numbers of turns are within the scope. The corkscrew 386 has a flat leading edge 394. Positioned at or near the first end 382 of the implant 380 is a hole for receiving sutures. An axial view of the flat corkscrew design of the implant 380 is also included by looking at the pointed tip 396 of the first end 382.

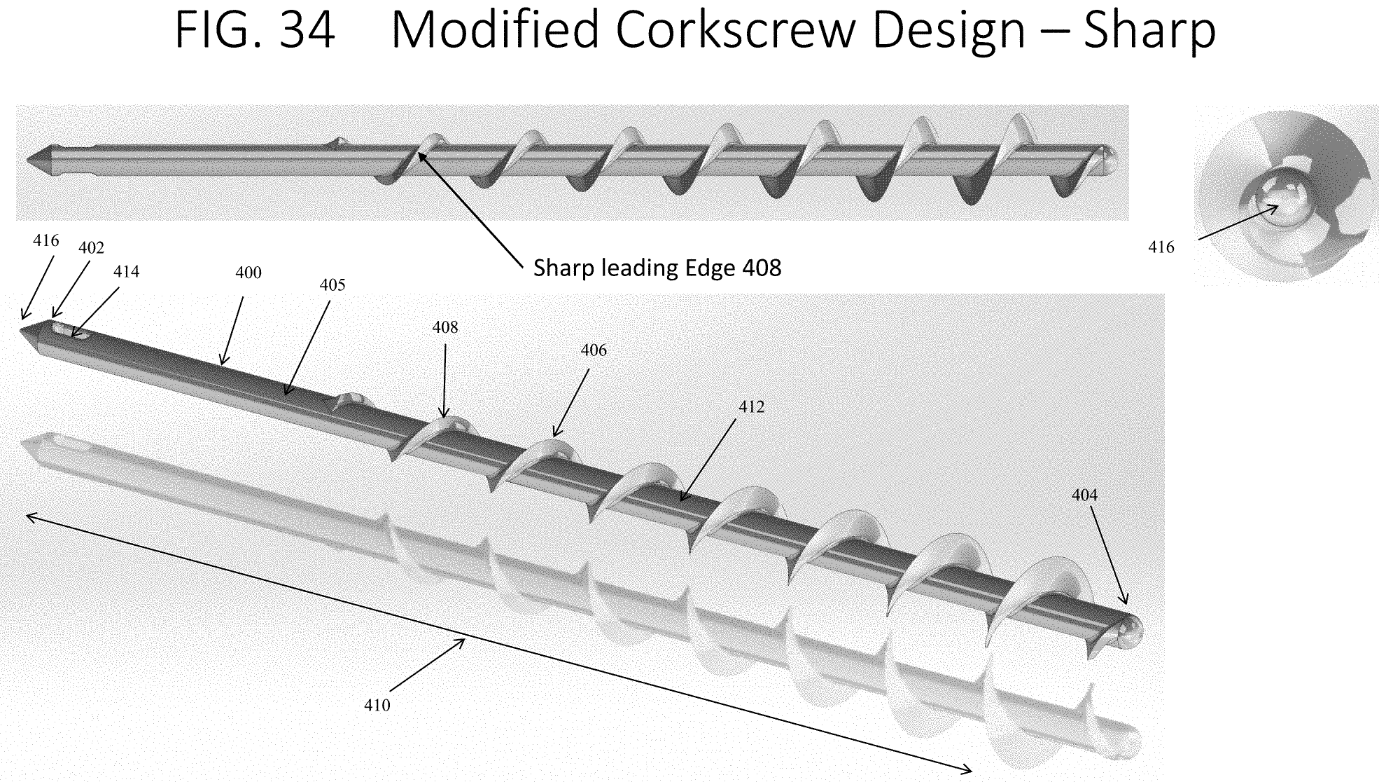

FIG. 34 shows an implant 400, which has a first end 402 and a second end 404 between an elongated rod like body 405 having a length 410 of approximately 35 mm, and a range of 25 mm to 50 mm is within the scope, but other lengths are within the scope of the invention. A pointed tip 416 is located at the first end 402 of the implant 400. A corkscrew 406 runs the length 410 of the implant 400 along its surface 412, with the number of corkscrew turns being eight turns, although other numbers of turns are within the scope. The corkscrew 406 has a sharp leading edge 408. Positioned at or near the first end 402 of the implant 400 is a hole 414 for receiving sutures. An axial view of the sharp corkscrew design of the implant 400 is also included by looking at the pointed tip 416 of the first end 402.

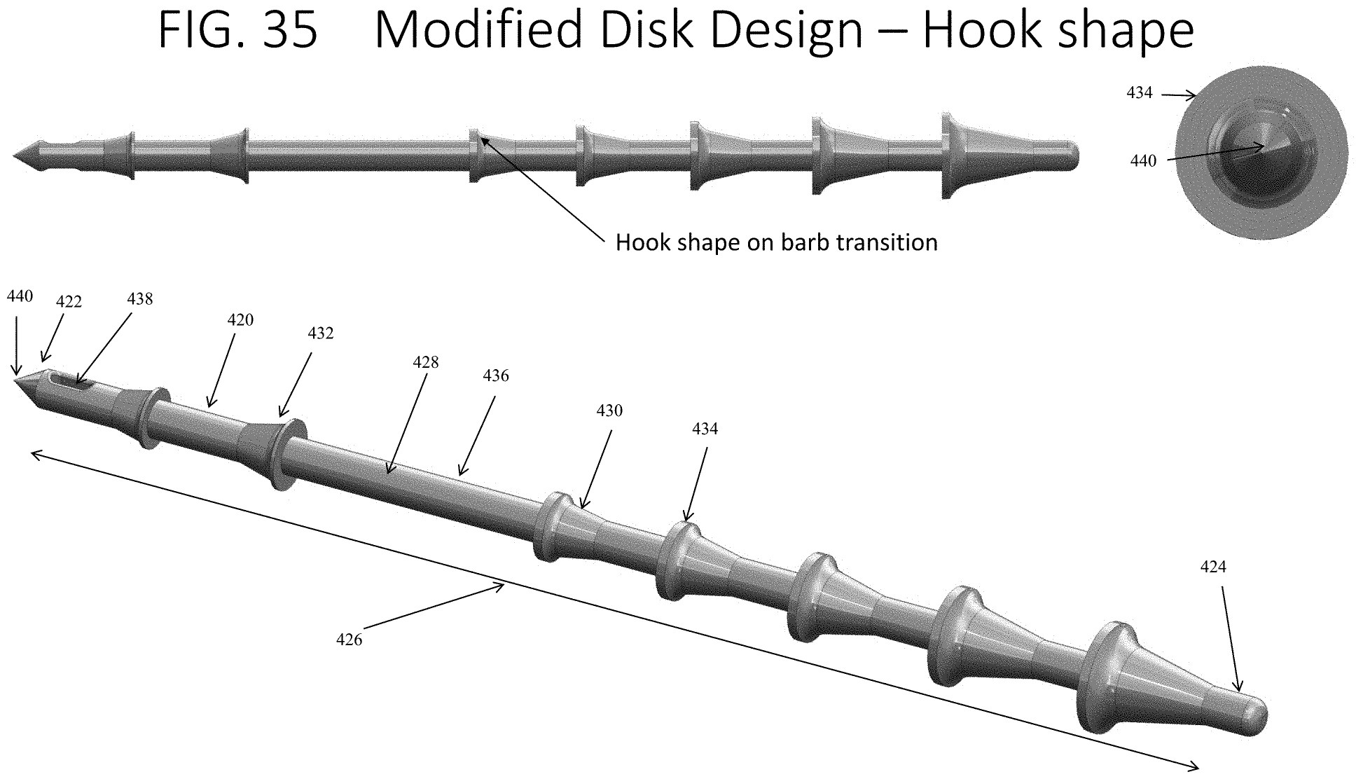

FIG. 35 shows a barbed implant 420 with disk hook shapes for the barbs, which has a first end 422 and a second end 424 between an elongated body 428 having a length 426 of approximately 35 mm, and a range of 25 mm to 50 mm is within the scope, but other lengths are within the scope of the invention. A pointed tip 440 is located at the first end 422 of the implant 420. A plurality of back facing hook barbs 430 are aligned along the body 428 of the implant 420, with five shown in FIG. 35. There are also one or more front facing hook barbs 432 positioned along the elongated body 428, with two front facing barbs shown. The barbs are disks 434 with a slight hook on the transition. Between the front facing hook barbs 432 and the back facing barbs 430 is an area 436 without barbs or hooks. Positioned at or near the first end 422 of the implant 420 is a hole 438 for receiving sutures. An axial view of the disk hook design 420 is also included by looking at the pointed tip 440 of the first end 422.

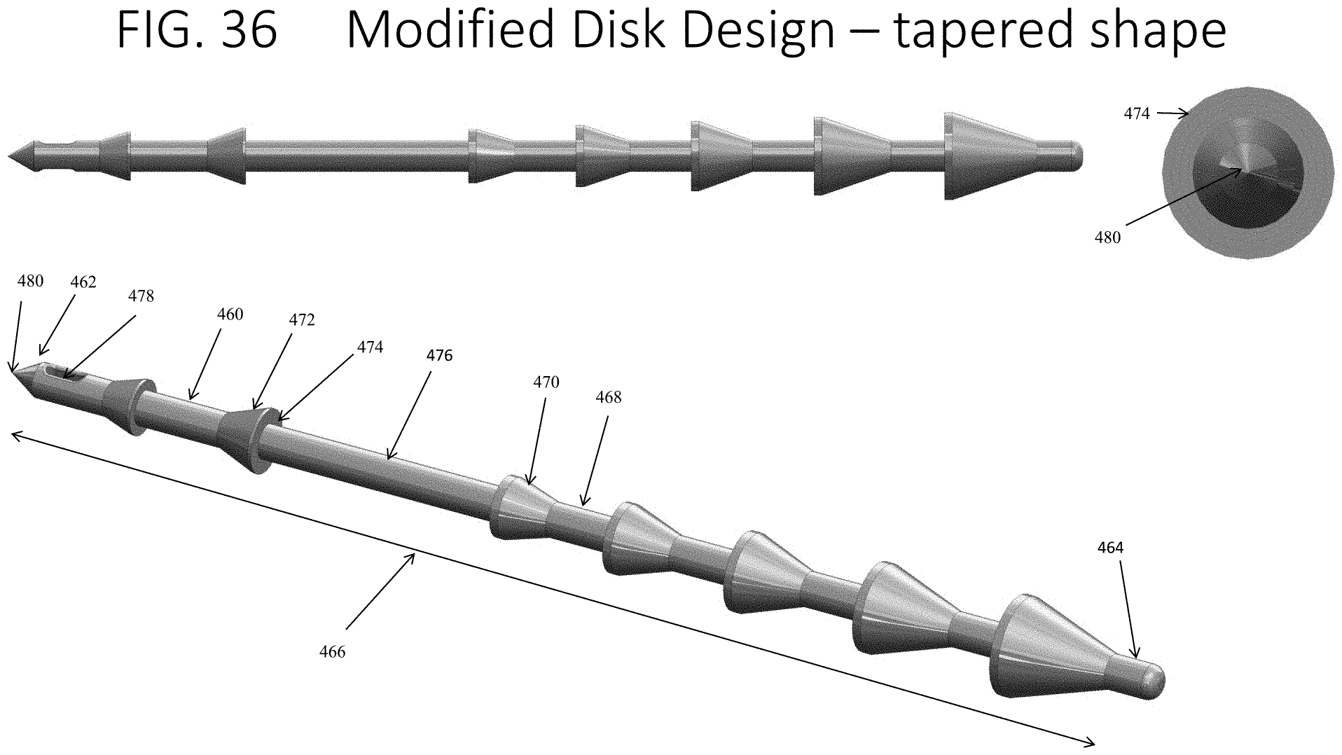

FIG. 36 shows another embodiment of a barbed implant 460 with disks that are tapered shapes for the barbs, which has a first end 462 and a second end 464 between an elongated body 468 having a length 466 of approximately 35 mm, and a range of 25 mm to 50 mm is within the scope, but other lengths are within the scope of the invention. A pointed tip 480 is located at the first end 462 of the implant 460. A plurality of back facing tapered disc barbs 470 are aligned along the body 468 of the implant 460, with five shown in FIG. 36. There are also one or more front facing tapered disk barbs 472 positioned along the elongated body 468, with two front facing disks barbs shown. The barbs are disks 474 with a tapered slant on the transition. Between the front facing disk barbs 472 and the back facing disk barbs 470 is an area 476 without disks or barbs or hooks. Positioned at or near the first end 462 of the implant 460 is a hole 478 for receiving sutures. An axial view of the tapered disk design implant 460 is also included by looking at the pointed tip 480 of the first end 462.

Studies—below are results of studies conducted.

1. Minimally Invasive Epiglottoplasty Using a Trans-Cervical Barbed Suture

Objective: Epiglottoplasty has been a reliable surgical intervention as a treatment for Obstructive Sleep Apnea (OSA) secondary to a displaced, retroflexed, inverted, floppy or otherwise obstructing epiglottis. Described is a new, minimally invasive technique without incision, using a trans-cervical insertion of a barbed suture to secure the epiglottis in a non-obstructing, anterior and upright position.

Methods: We treated 6 patients with known OSA and findings consistent with an obstructing epiglottis. All patients underwent a minimally invasive procedure including transoral robotic (or endoscopic) demucosalization of the dorsal surface of the epiglottis as well as the corresponding surface of the base of tongue. Next, a loaded suture passer is placed trans-cervically, midline, just below the hyoid and passed through the base of tongue. A barbed suture is then guided robotically (or endoscopically) through the epiglottis. A second pass is placed above the hyoid, the suture is grasped with suture passer and withdrawn through the neck. Tension applied on the suture positions the epiglottis to the base of tongue.

Results: At the 3 month, post-operative visit all 6 patients had improvement in Polysomnogram, Epworth Sleepiness Scale, and Pittsburgh Sleep Quality Index, indicating both subjective and objective improvement in their sleep quality.

Conclusion: A displaced epiglottis can be an underappreciated cause of OSA. Epiglottoplasty has been a mainstay of treatment for this type of obstruction. We propose a new minimally invasive method of epiglottoplasty using a trans-cervical barbed suture, without external incisions. Both subjective and objective post-operative results have been encouraging.

2. Robotic Assisted Minimally Invasive Suspension of Hyoid with or without Epiglottoplasty

Objectives: At the conclusion of this presentation the participants should be able to understand how to select surgical candidates and perform minimally invasive hyoid suspension and epiglottoplasty to correct floppy epiglottis and tongue base collapse, utilizing flex robotic instrumentation.

Study Design Retrospective Cohort

Introduction: OSA is a common disorder effecting almost 50 million people. Although CPAP is considered the gold standard therapy for OSA, almost 50% of patients are non-compliant. Surgical options to correct tongue base or epiglottic collapse are considered invasive with significant morbidity. We introduced an effective procedure addressing hypopharyngeal collapse without external incisions.

Methods: Fifty two patients underwent robotic assisted suspension of hyoid with or without epiglottoplasty over the last 3 years. When floppy or folded epiglottis was present with tongue collapse, an epiglottoplasty also was included to hyoid suspension. Flex robot and instrumentation were utilized allowing exposure, great visualization to safely perform this minimally invasive procedure. Resorbable, heavy barbed sutures and suture passer allows successful completion without incision or tissue resection. When indicated minor ablative steps can also be combined such as lingual tonsil ablation.

Results: Over the last three years 52 patients undergone hyoid suspension, 37 were male and 15 female, mean age was 43. Twenty six patients had additional tongue base or epiglottic procedures. Overall surgical time including robotic set up, was under one hour. Since this procedure is performed in the central, neurovascular safe zone, no surgical complications were recorded. Majority of the patients were discharged same day with minimal discomfort, tolerating oral diet. Post-operative sleep parameters improved in all patients, including, AHI (41%), ODI (52%), FOQ (60%) and Epworth scale (60%).

Conclusions: A minimally invasive surgical method utilizing Flex robot and barbed sutures for hypopharyngeal obstruction is introduced. Robotic access allows the procedure to be performed without external incisions. This represents new and effective method for OSA patients secondary to tongue or epiglottic collapse who are not CPAP compliant.

While illustrative embodiments of the invention have been described above, it is, of course, understood that many and various modifications will be apparent to those of ordinary skill in the relevant art, or may become apparent as the art develops. Such modifications are contemplated as being within the spirit and scope of the invention or inventions disclosed in this specification.

What is claimed is:

1. A method for enlarging an airway of a patient comprising:
inserting a suture passer device loaded with a suture into a patient at a first position; said patient have a neck and hyoid bone;
said first position located on the neck of said patient over said hyoid bone;
said suture passer device comprising,
an elongated needle like instrument having a body with an external surface, a first end received by a handle section and a second end with a needle tip;
said instrument having an insertion hook for securing said suture for insertion and an extraction hook for securing said suture during extraction;
said insertion hook and said extraction hook defined by an open mouth area located between said insertion hook and said extraction hook, and an internal surface extending from the external surface of the body adjacent to said insertion hook to said external surface of the body adjacent to said extraction hook to form an opening in the body of said suture passer device which receives the suture when said suture is placed into said mouth area between said insertion hook and said extraction hook;
said insertion hook securing said suture for delivering an insertion of said suture by said instrument and said extraction hook securing said suture for extraction of said suture by said instrument
said suture loaded on said suture passer device on said insertion hook;
said needle tip of said suture passer device loaded with said suture piercing tissue inside said patient to create a first opening;
extending said suture passer device with said suture on said insertion hook through said first opening and passing said suture over said hyoid bone and into a throat cavity and airway of said patient;
releasing said suture from said insertion hook of said suture passer device;
removing said suture passer device through said first opening;
moving said needle tip of said suture passer device to a second position under said hyoid bone;
inserting said suture passer device through a second tissue location to create a second opening, with said extraction hook extending through said second opening;
reloading said suture onto said suture passer device on said extraction hook;
extracting said suture passer device with said suture through said second opening to form a suture loop extending from said first opening through said second opening, said suture loop looping around said hyoid bone;
pulling said suture loop upward through said first and second opening to suspend said tissue and epiglottis tissue upward and enlarge said throat cavity and said airway of the patient;
fixing said suture externally on said neck of said patient.

2. The method according to claim 1 wherein said suture is a barbed suture.

3. The method according to claim 1 further comprising suspending the hyoid bone of the patient and thereby opening the airway of said patient.

4. The method according to claim 1 further comprising suspending tongue base tissue of the patient and thereby opening the airway of said patient.

5. The method according to claim 1 further comprising suspending thyroid of the patient and thereby opening the airway of said patient.

6. The method according to claim 1 wherein suspension of tissue is accomplished by a barbed suture composed of an absorbable material.

7. The method according to claim 1 wherein said instrument of said suture passer device includes a head section angled away from a central axis of said instrument body.

8. The method according to claim 1 wherein said insertion hook of said suture passer device is located on said instrument at a position proximal to said handle section relative to said extraction hook.

9. The method according to claim 1 wherein said suture passer device is used in procedures of robotic assisted minimally invasive suspension of a hyoid.

10. The method according to claim 1 wherein said step of releasing said suture from said insertion hook is by use of forceps of a robotic system grasping said suture and releasing said suture from said suture passer.

11. The method according to claim 1 wherein said step of reloading said suture onto said extraction hook is by forceps of a robotic system loading said suture on said extraction hook.

12. The method according to claim 1 wherein said suture passer device is used in procedures with a camera.

13. The method according to claim 1 wherein said needle tip extends past a patient's epiglottis after piercing said tissue inside said patient to create said first opening.

14. The method according to claim 1 wherein said step of extending said suture passer device with said suture on said insertion hook through said first opening and passing said suture over said hyoid bone further includes extending said suture passer device over a lingual tonsil area in said first position and said step of moving said needle tip is behind said lingual tonsil area in said second position.

15. The method according to claim 3 wherein suspending the hyoid bone includes suspension of epiglottis tissue.

16. The method according to claim 4 wherein suspending the tongue base tissue includes suspension of epiglottis tissue.

17. The method according to claim 2 wherein said barbed suture is a No. 2 barbed suture.

18. The method according to claim 1 wherein said suture is an implant.

19. The method according to claim 18 wherein said implant is a barbed implant.

20. The method according to claim 19 wherein said barbed implant is a barbed suture.

21. The method according to claim 19 wherein said barbed implant is a shaped barbed implant.

22. The method according to claim 18 wherein said implant is a suture like device.

23. The method according to claim 20 wherein said barbed suture is a bidirectional barbed suture.

24. The method according to claim 19 wherein said barbed implant is absorbable.

25. The method according to claim 1 wherein said suture passer device has at least one of said insertion hook or said extraction hook marked for visual aid.

26. The method according to claim 19 wherein said barbed implant has a body with back facing barbs and front facing barbs.

27. The method according to claim 19 wherein said barbed implant has a body with a corkscrew along said body for barbs.

28. The method according to claim 19 wherein said barbed implant has a body with disk shapes for barbs.
